(19) United States
(12) Patent Application Publication
(10) Pub. No.: US 2008/0220105 A1
(43) Pub. Date: Sep. 11, 2008

(54) Composition Containing Lotus Extract

(75) Inventor: Suping Jiang, Tokyo (JP)

Correspondence Address: WENDEROTH, LIND & PONACK, L.L.P., 2033 K STREET N. W., SUITE 800, WASHINGTON, DC, 20006-1021, US

(73) Assignee: YUGEN KAISHA KONNO INTERNATIONAL, Tokyo (JP)

(21) Appl. No.: 11/793,212

(22) PCT Filed: Oct. 12, 2005
(86) PCT No.: PCT/JP05/18786

§ 371 Date: Jun. 18, 2007

(30) Foreign Application Priority Data
Dec. 24, 2004 (JP) 2004-373647

(51) Int. Cl. A61K 36/00; A23L 1/238; A23L 1/39; A23L 1/22; A23L 1/10

(52) U.S. Cl. 424/774; 426/655; 426/589; 426/634; 426/638; 426/549; 426/660; 426/618; 424/778; 424/779

(57) ABSTRACT

For attaining effective utilization of lotus petals, receptacles, stem, leaves and husks having been discarded, there is provided a composition comprising an extract from at least one selected from among lotus petal, receptacle, stem, leaf and husk wherein anthocyanidin, various minerals and vitamins are contained in high proportion. Addition of this composition to various processed foods would provide processed foods useful for the health maintenance of eyes. Addition of this composition to various cosmetics would provide cosmetics useful for promotion of blood circulation and cell activation.

### TECHNICAL FIELD

The present invention relates to a composition containing an extract from a lotus “Nelumbo” (a perennial plant of Nymphaeaceae) that is cultivated in a pond, a march or a rice field to harvest its root and fruit.

### BACKGROUND ART

A lotus has been utilized as an herbal drug from the past, and in particular, its leaves are used as a “tea” drink and are said to be effective for lowering blood sugar level and blood pressure. It is widely known that lotus roots and lotus fruits are used as food materials. However, in lotus cultivation, lotus roots and lotus fruits are harvested, but the other parts are almost all discarded. However, it has been known that the other parts of a lotus, for example, its petal, receptacle, stem (including flower stalk and leaf stem), leaf, and skin to cover the fruit thereof (hereinafter this is referred to as “husk”) contain a large amount of “anthocyanidin” that is a nourishment ingredient for eyes, and that the edible lotus fruit and root contain many minerals and a large amount of vitamin C.

Further, many plants that are called “lotus” are known, and extracts from some of them are known to be effective for lowering blood sugar level and for promoting collagen production, and are used as ingredients of drugs, cosmetics, or foods and drinks for medical care or for beauty.

However, there are many varieties of plants called “lotus”, and they differ in point of their naming and classification (genus), therefore necessarily differing in point of their constitutive ingredients. Even in a lotus of the same variety, its constitutive ingredients may differ in different sites of fruit, root, stem, leaf and flower.

Regarding a lotus fruit utilized as a food material, those disclosed in JP-A 2000-69936 are known. The known food material is such that a lotus fruit powder is added to a health food that essentially comprises a black dietary powder of black beans, black sesame, brown rice or the like, as the third component thereof in such a manner that the resulting composition may be neutral having a good plus/minus balance in Chinese medicine theory; and when it is taken, side effects such as blood pressure rise, body temperature rise and sweating are suppressed.

Though differing from a lotus Nelumbo of Nymphaeaceae in point of its variety, a blood sugar level depressor that comprises an extract of “Sinocrassula indica (Decne.) Berger” and “Sinocrassula indica (Decne.) Berger var. obtusffolla (Frod.) S. H. Fu.”, which is said to have a blood sugar level-depressing effect, and a blood sugar level-depressing food that comprises it are known as disclosed in JP-A 2000-128797. Further, as its potency, the extract has an antpyretc and detoxifying effect, and a hemostatic and antidiarrheal effect, and is therefore used for treatment of throat tumor, bacterial diarrhea, uterine hemorrhage and hematochezia. In particular, it is said that the flowers and the leaves of the plant are most favorably used.

Further, also though differing from a lotus Nelumbo of Nymphaeaceae in point of its variety, a collagen production promoter, a skin cosmetic, and a food and a drink for beauty that comprise an extract from “Polygalafallax” and “Altemanthera sessilis”, which is said to have a collagen production-promoting effect, are known as disclosed in JP-A 2003-212748. The extracts from these plants are any of a liquid extract, a diluted or concentrated liquid of the liquid extract, a dried product obtained by drying the liquid extract, or their roughly-purified or fully-purified products; and the compositions may contain any of them as the active ingredient thereof. They say that branches of “Polygalafallax” and leaves of “Altemanthera sessilis” are especially preferably used for extraction.

However, in JP-A 2000-69936 mentioned above, lotus fruits are used as powder, and the powder is necessarily incorporated as a side ingredient to the black dietary powder serving as the essential ingredient, and this is used for neutralization of the resulting composition with the plus/minus balance thereof in Chinese medicine theory taken into consideration; and the potency of the ingredient of the lotus fruit itself is not so much expected.

The “Sinocrassula indica (Decne.) Berger” and “Sinocrassula indica (Decne.) Berger var. obtuslfolla (Frod.) S. H. Fu.” in the known technique of JP-A2000-128797 mentioned above are biennial herb plants belonging to Crassulaceae, and they grow on the rocks over low mountains and high mountains, and it is recognized that they are substantially different plants from a lotus “Nelumbo” (a perennial plant of Nymphaeaceae) that is cultivated in ponds, marches or rice fields. Regarding their sites for use, it is said that their flowers and leaves are preferred, and therefore they are obviously different from a lotus in point of their ingredients and potency.

Further, “Polygalafallax” in the known technique of JP-A 2003-212748 mentioned above is a shrub belonging to Polygalaceae, and “Altemanthera sessilis” is an annual herb belonging to Amaranthaceae, and it is considered that these are both substantially different plants from a lotus “Nelumbo” (a perennial plant of Nymphaeaceae) that is cultivated in ponds, marches or rice fields. Regarding their sites for use, it is said that branches of “Polygalafallax” and leaves of “Affemanthera sessilis” are especially preferably used for extraction; and therefore they are obviously different from a lotus in point of their ingredients and potency.

On the other hand, regarding the lotus “Nelumbo” belonging to Nymphaeaceae, its petal, receptacle, stem, leaf and husk except its root and fruit have heretofore been discarded.

### DISCLOSURE OF INVENTION

Accordingly, it is an object of the invention to effectively utilize the lotus petal, receptacle, stem, leaf and husk that have heretofore been discarded.

The present inventor has found that the petal, receptacle, stem, leaf and husk of a lotus contain useful ingredients such as anthocyanidin that is a nourishment ingredient for eyes, as well as various minerals and vitamins contributing to activation of cells, and has assiduously studied and completed the present invention. Specifically, the inventor has created a composition containing a necessary amount of an extract from at least one of lotus petals, receptacles, stems, leaves and husks.

The extract may be powdery or liquid. Regarding its use, the above composition may be incorporated into various food materials to produce processed foods for daily life, and may be therefore helpful for function recovery of eyes. In this case, the extract content is preferably within a range of from 0.1 to 5.0 parts by weight relative to 100 parts by weight of the food material. The processed foods include various breads such as loaves of bread; confectionery such as Japanese sweets, western-style cakes; seasonings such as miso (soybean paste), soy sauce, sauce for Korean barbecue, Worcester sauce, curry roux; noodles such as lamian (Chinese noodles in soup), udon (wheat noodles), soba (buckwheat noodles).

Further, cosmetics containing the composition may be effective for cell activation owing to various minerals and vitamins therein. The cosmetics are preferably emulsions, creams or powders.

The composition of the invention comprises a necessary amount of an extract from at least one of lotus petals, receptacles, stems, leaves and husks, and the extract contains a large amount of anthocyanidin that is a nourishment ingredient for eyes. Accordingly, by incorporating it into processed foods much eaten in daily life, for example, various breads such as loaves of bread; confectionery such as Japanese sweets, Western-style cakes; seasonings such as miso (soybean paste), soy sauce, sauce for Korean barbecue, Worcester sauce, curry roux; noodles such as lamian (Chinese noodles in soup), udon (wheat noodles), soba (buckwheat noodles), anyone may take a nourishment ingredient for eyes everyday, though by a small quantity thereof; and, in addition, by incorporating a small quantity of it into suitable tablets for vitamin intake, or nutritional supplements for calcium and magnesium intake, the composition exhibits an excellent effect in that it greatly contributes to various health foods.

The composition of the invention contains minerals such as potassium, calcium, magnesium, phosphorus, and also vitamin C, in addition to anthocyanidin, and therefore contributes to activation of cells. Accordingly, by incorporating it in cosmetics, it exhibits an excellent effect of contributing to fair skin formation.

### BEST MODE FOR CARRYING OUT THE INVENTION

The invention is described in detail hereinunder with reference to its concrete embodiments.

First, the lotus extract powder to be contained in, or that is, to be added to or incorporated in the composition of the invention is described roughly in point of the parts of a lotus to be used therein and as to the method of extraction. In general, during lotus cultivation in a lotus field or a lotus marsh, lotus roots are harvested in an ordinary manner; and before lotus roots are harvested, lotus leaves, stems and flower stalks along with their receptacles are cut out from the parts near their roots, then washed with water to remove the mud adhering thereto make them clean. After flower stalks are cut off from receptacles, lotus fruits are removed. Lotus flowers are cut out along with their flower stalks before they bear fruits, and processed for extraction.

Lotus leaves, receptacles from which stems (including leaf stems and flower stalks) and fruits are removed, and husks to cover fruits are finely pulverized and put into water, and after a few (from 3 to 5) hours, the pulverized mixture is squeezed along with water to remove the solid matter. In this case, the pulverized mixture is put into water, and this is because the nourishment ingredients such as anthocyanidin, minerals and vitamin C to be extracted are soluble in water, and by putting the pulverized mixture into water, these nourishment ingredients may be dissolved out into water and may be efficiently extracted within a short period of time.

Then, the solution from which the solid matter has been removed is kept statically as it is, whereby any further finer solid matter is precipitated, and only the supernatant part is dewatered according to a suitable method thereby to make the extract left as a powder or as a liquid from which a major part of water is removed off. Regarding the dewatering method, for example, the supernatant may be heated so as to remove water through vaporization, or may be frozen also to remove water from it.

As other extraction methods, a solvent for extraction such as alcohol may be used. Also in this case, it is desirable that the leaves, the stems (including leaf stems and flower stalks), the receptacles and the husks are pulverized or cut into small chips for efficient extraction. In case where a solvent for extraction such as alcohol is used, efficient extraction may be attained even within a short period of extraction time of, for example, 1 or 2 hours, and separation of the extract from the extraction solvent is easy. In short, any conventional known method may be employed for extraction. The obtained extract may be either powdery or liquid, and the obtained extract is nearly dark brown as a whole and tastes bitter.

The content of the extract ingredients, anthocyanidin, minerals and vitamin C in different parts of a lotus is computed through absorptiometry, using 1% delphinidin (hydrochloric acid/methanol=2:98 solution) at a molar absorption coefficient of 1020, and the resulting data are shown in Table 1 below.

TABLE 1

| | Anthocyanidin | Minerals | Vitamin C |
|---|---|---|---|
| Parts of Lotus | [mg/g] | [mg/g] | [mg/g] |
| petal | 6.66 | 4.30 | 15.17 |
| receptacle | 7.54 | 12.74 | 8.11 |
| leaf | 7.97 | 11.23 | 18.02 |
| flower stalk | 2.17 | 20.54 | 14.66 |
| leaf stem | 1.50 | 20.40 | 14.35 |
| fruit | 0.10 | 18.32 | 20.23 |
| root stem (lotus root) | 0.07 | 30.10 | 16.71 |
| husk | 4.52 | 19.41 | 15.84 |

As is obvious from the above Table 1, it is confirmed that lotus petals, leaves spectacles stems and husks contain not only anthocyanidin but also a large quantity of minerals and vitamin. Heretofore, lotus roots and fruits are harvested; but naturally, the other parts are discarded as such. However, it has been clarified that the parts to be discarded can be effectively utilized. In particular, it has been clarified that the content of anthocyanidin in those parts is about 7 times that of anthocyanidin (3.3 mg/g) in wild blueberry.

Next, loaves of bread were produced, containing the extract powder obtained in the manner as above. The extract powder was added to a dough powder in an amount of about 0.1 to 5.0 parts by weight relative to 100 parts by weight of the dough powder, thereby preparing dough. In this step, it is desirable that the extract powder is dissolved in cold or warm water to be added for kneading dough, and is added to and mixed with a dough powder before the dough is kneaded, since the powder may be uniformly mixed in the dough as a whole. The extract that is liquid may be dissolved in cold or warm water so as to be diluted as a whole before use.

When the amount of the extract (powdery or liquid) is more than 5.0 parts by weight relative to 100 parts by weight of the dough powder, then the bread tastes somewhat bitter. When the amount of the extract is less than 0.1 parts by weight, then the effect of the nourishment ingredient may decrease and the meaning of the composition as a health food may lower. Therefore, it is desirable that the extract powder is incorporated in a dough powder in an amount of about from 0.1 to 5.0 parts by weight relative to 100 parts by weight of the dough powder. Different from vitamins, anthocyanidin is resistant to heat and its potency does not change even when heated or baked, and therefore it has been known that, even in confectionery, for example, Japanese sweets, western-style cakes, and snack cakes such as biscuits and rice crackers, the extract may be incorporated in the same manner and in the same ratio as above, and those confectionery may be produced in an ordinary manner.

In addition, since the extract is hardly degraded through processing such as heating or baking, it may be added to any other various processed foods, for example, seasonings such as miso, soy sauce, sauce for Korean barbecue, Worcester sauce, curry roux; noodles such as lamian (Chinese noodles in soup), udon (wheat noodles), soba (buckwheat noodles), and others. In any case, it is desirable that the amount of the extract is within the above-mentioned range. Needless-to-say, in addition, the extract may be used in feed materials for pets.

For foods that are required not to be so much colored, for example, for loaves of bread or for noodles, the amount of the extract to be added may be reduced so that the foods are not so much discolored as a whole. On the other hand, for seasonings such as miso and soy sauce, the extract may be added to them with not so much specific attention to be paid for their discoloration and bitterness; and therefore, its amount may be increased in some degree (up to about 10 parts by weight) with no trouble. Since the amount of the seasoning in general use may be small, the extract may not change the taste of the food materials to which it is added. Moreover, since the extract is soluble in water, it may be added to the products during their production, or may be added to and mixed with them after their production.

The minerals and vitamin C that exit in the extract along with anthocyanidin therein are effective for cell activation owing to their blood flow-promoting effect, and therefore, when the extract is added to or incorporated in various cosmetics in a suitable amount (from about 0.1 to 1.0%) thereof, then it may be absorbed through a skin to thereby improve the function of the cosmetics for skins. Further, when the extract is added to or incorporated in a predetermined amount (from about 1% to about 10%) in a masking material (agent) that is used in beauty salons such as aesthetic treatment salons for skin rejuvenation, then it may act to further promote blood flows for cell activation.

### INDUSTRIAL APPLICABILITY

In the manner as above, a composition containing an extract (powdery or liquid) from lotus petals, receptacles, stems leaves and/or husks contains much anthocyanidin that is a nourishment ingredient for eyes, and therefore it is useful as an additive to drinks and foods that are to be taken into bodies. Accordingly, when it is taken everyday, it greatly contributes to eye health maintenance, and may be utilized in various processed foods and supplements.

In addition, the minerals and vitamin C existing in the extract along with anthocyanidin therein have a blood flow-promoting effect for cell activation, and therefore the extract may be used in various cosmetics.

1. A composition containing a necessary amount of an extract from at least one of lotus petals, receptacles, stems, leaves and husks.

2. The composition as claimed in claim 1, wherein the extract is powdery or liquid.

3. A processed food containing the composition of claim 1.

4. The processed food as claimed in claim 3, wherein the extract content is within a range of from 0.1 to 5.0 parts by weight relative to 100 parts by weight of the food material.

5. The processed food as claimed in claim 3, which is any of breads such as loaves of bread; confectionery such as Japanese sweets, western-style cakes; seasonings such as miso (soybean paste), soy sauce, sauce for Korean barbecue, Worcester sauce, curry roux; noodles such as lamian (Chinese noodles in soup), udon (wheat noodles), soba (buckwheat noodles).

6. A cosmetic containing the composition of claim 1.

7. The cosmetic as claimed in claim 6, which is any of emulsions, creams or powders.

8. A processed food containing the composition of claim 2.

9. The processed food as claimed in claim 8, wherein the extract content is within a range of from 0.1 to 5.0 parts by weight relative to 100 parts by weight of the food material.

10. The processed food as claimed in claim 8, which is any of breads such as loaves of bread; confectionery such as Japanese sweets, western-style cakes; seasonings such as miso (soybean paste), soy sauce, sauce for Korean barbecue, Worcester sauce, curry roux; noodles such as lamian (Chinese noodles in soup), udon (wheat noodles), soba (buckwheat noodles).

11. A cosmetic containing the composition of claim 2.

12. The cosmetic as claimed in claim 11, which is any of emulsions, creams or powders.
